(19) United States
(12) Patent Application Publication
(10) Pub. No.: US 2012/0172476 A1
(43) Pub. Date: Jul. 5, 2012

(54) POLYURETHANE FOAMING PROCESSES AND FOAM PROPERTIES USING HALOGENATED OLEFIN BLOWING AGENT

(75) Inventors: Joseph S. Costa, Gilbertsville, PA (US); Benjamin Bin Chen, Wayne, PA (US); Philippe Bonnet, Lyon (FR)

(73) Assignee: Arkema Inc., King of Prussia, PA (US)

(21) Appl. No.: 13/394,355

(22) PCT Filed: Sep. 1, 2010
(86) PCT No.: PCT/US2010/047453

§ 371 Date: Mar. 6, 2012

Related U.S. Application Data
(60) Provisional application No. 61/240,727, filed on Sep. 9, 2009.

(51) Int. Cl. C08G 18/06; C08K 5/01; C08K 5/05; C08J 9/06; C08K 5/06; C08K 3/20; C07C 21/18; C08K 5/00; C08K 5/07

(52) U.S. Cl. 521/170; 106/499; 570/135; 264/54

(57) ABSTRACT

The present invention relates to a method of producing more uniformly distributed polyurethane form using blowing agents. More particularly, the present invention relates to a method of producing more uniformly distributed polyurethane foam for an application in which flow of liquid polyurethane foam prior to solidification is important to its performance using a hydrochlorofluoroolefin (HCFO), such as 1233zd.

### FIELD OF THE INVENTION

The present invention relates to a method of producing more uniformly distributed polyurethane foam using blowing agents. More particularly, the present invention relates to a method of producing more uniformly distributed polyurethane foam for an application in which flow of liquid polyurethane foam prior to solidification is important to its performance using the hydrochlorofluoroolefin (HCFO), such as 1233zd.

### BACKGROUND OF THE INVENTION

The Montreal Protocol for the protection of the ozone layer, signed in October 1987, mandated the phase out of the use of chlorofluorocarbons (CFCs). Materials more “friendly” to the ozone layer, such as hydrofluorocarbons (HFCs) eg HFC-134a replaced chlorofluorocarbons. The latter compounds have proven to be green house gases, causing global warming and were regulated by the Kyoto Protocol on Climate Change, signed in 1998. The emerging replacement materials, hydrofluoropropenes, were shown to be environmentally acceptable i.e. has zero ozone depletion potential (ODP) and acceptable low global warming potential (GWP).

Currently used blowing agents for polyurethane forms include HFC-134a, HFC-245fa, HFC-365mfc that have relatively high global warming potential, and hydrocarbons such as pentane isomers which are flammable and have low energy efficiency. Therefore, new alternative blowing agents are being sought. Halogenated hydroolefinic materials such as hydrofluoropropenes and/or hydrochlorofluoropropenes have generated interest as replacements for HFCs. The inherent chemical instability of these materials in the lower atmosphere provides the low global warming potential and zero or near zero ozone depletion properties desired.

Polyurethane foam processing conditions have a profound impact on the properties of foam. Density and thermal conductivity variations, compression strength etc are key parameters for the performance of the product. Arai et al Proceedings of the SPI-29^{th} 1995, p. 272 showed that foam density was affected by the pressure variation. Lefebvre et al Int. J. Numer Methods Fluids 1995, vol 20, p. 319 claimed that foam density was related to temperature of the exothermic reaction. The self-expanding fluid has significantly different flow behaviors from that of the Newtonian fluid with relatively constant density. Mitani et al Polym. Eng. Sci 2003 vol 43(9), p. 1603 used three-dimensional control volume finite element method to solve the Stokes equations under isothermal conditions. The density change was predicted by assuming the density was a function of time. US2008/0255262 disclosed a method of molding rigid polyurethane foams with enhanced thermal conductivity under reduced pressure.

The object of the present invention is to provide a method of using compositions comprising hydrohaloolefins, in particular, 1-chloro-3,3,3-trifluoropropene-1 (HCFO-1233zd) for polyurethane foams that provides improved processability and foam properties that are related to thermal insulation.

### BRIEF DESCRIPTION OF THE DRAWINGS

FIG. 1 is a graph of foam density versus distribution within the panel.

### DESCRIPTION OF THE INVENTION

The present invention relates to a method of generating liquid polyurethane foams that have unexpectedly uniform density distribution along their flow pathway before they are solidified and enhance processing efficacy. According to one embodiment, the present invention comprises 1) mixing the blowing agent with other polyurethane premix components; 2) then using high pressure mixing and dispensing equipment of reaction injection molding.

The foam processing efficacy was characterized by minimum fill weight in a mold, core density, average density and density distribution in the flow path, compression strength of foam, dimensional stability and thermal conductivity of foams.

The present invention is directed towards using blowing agents with negligible (low or zero) ozone-depletion and low GWP based upon unsaturated halogenated hydroolefins in combination with polyol(s), silicone surfactant(s), amine catalyst(s), carbon dioxide generating agent(s), and other(s).

The blowing agent comprises an unsaturated halogenated hydroolefin such as hydrofluoroolefins, hydrochlorofluoroolefins, and the like, in particular, predominately trans or E-1233zd, 1-chloro-3,3,3-trifluoropropene alone or in a combination including a hydrofluoroolefin (HFO), a hydrochlorofluoroolefin (HCFO) not including 1233zd, a hydrofluorocarbon (HFC), a hydrofluoroether (HFE), a hydrocarbon, an alcohol, an aldehyde, a ketone, an ether/diether or carbon dioxide. It was found that liquid polyurethane foam prior to solidification flowed more uniformly than others, which is surprising based on its boiling point and relative solubility in the polymer premix. The resulted polymer along the flow path showed much narrower density variation defined by overall minus core density, from 0.10 to 0.65 pound per cubic feet (pcf), preferably 0.15 to 0.50 pcf, and even more preferably from 0.20 to 0.45 pcf.

The preferred hydrofluoroolefins (HFO) typically contain 3, 4, or 5 carbons, and include but are not limited to pentafluoropropenes, such as 1,2,3,3,3-pentafluoropropene (HFO 1225ye), tetrafluoropropene, such as 1,3,3,3-tetrafluoropropene (HFO 1234ze), 2,3,3,3-tetrafluoropropene (HFO 1234)yf), 1,2,3,3-tetrafluoropropene (HFO1234ye), trifluoropropene, such as 3,3,3-trifluoropropene (1243zf), all tetrafluorobutenes (HFO 1345), all pentafluorobutene isomers (HFO1354), all hexafluorobutene isomers (HFO1336), all heptafluorobutene isomers (HFO1327), all heptafluoropentene isomers (HFO1447), all octafluoropentene isomers (HFO1438), all nonafluoropentene isomers (HFO1429). HCFOs such as, 1-chloro-3,3,3-trifluoropropenen (HCFO-1233zd), 2-chloro-3,3,3-trifluoropropene (HCFO-1233xf) and HCFO1223. Preferred embodiments of the invention are blowing agent compositions of unsaturated halogenated hydroolefins with normal boiling points less than about 60° C.

The blowing agents comprise a hydrohaloolefin such as hydrofluoroolefin, hydrochlorofluoroolefin, and the like, in particular, predominately trans or E-1233zd, 1-chloro-3,3,3-trifluoropropene alone or in combination with other blowing agents including (I) hydrofluorocarbons including but not limited to: difluoromethane (HFC32); 1,1,1,2,2-pentafluoroethane (HFC125); 1,1,1-trifluoroethane (HFC143a); 1,1,2,2-tetrafluorothane (HFC134); 1,1,1,2-tetrafluoroethane (HFC134a); 1,1-difluoroethane (HFC152a), 1,1,1,2,3,3,3-heptafluoropropane (HFC227ea); 1,1,1,3,3-pentafluopropane (HFC245fa); 1,1,1,3,3-pentafluobutane (HFC365mfc) and 1,1,1,2,2,3,4,5,5,5-decafluoropentane (HFC4310mee); (II) hydrofluoroolefins including but not limited to tetrafluoropropenes (HFO1234), trifluoropropenes (HFO1243), all tetrafluorobutenes (HFO 1345), all pentafluorobutene isomers (HFO1354), all hexafluorobutene isomers (HFO1336), all heptafluorobutene isomers (HFO1327), all heptafluoropentene isomers (HFO1447), all octafluoropentene isomers (HFO1438), all nonafluoropentene isomers (HFO1429); (III) hydrocarbons including but not limited to, pentane isomers, butane isomers; (IV) Hydrofluoroether (HFE) such as, C₄F₉OCH₃ (HFE-7100), C₄F₉OC₂H₅ (HFE-7200), CF₃CF₂OCH₃ (HFE-245cb2), CF₃CH₂CHF₂ (HFE-245fa), CF₃CH₂OCF₃ (HFE-236fa), C₃F₇OCH₃ (HFE-7000), 2-trifluoromethyl-3-ethoxydodecofluorohexane (HFE 7500), 1,1,1,2,3-hexafluoro-4-(1,1,2,3,3,3-hexafluoropropoxy)-pentane (HFE-7600), 1,1,1,2,2,3,5,5,5-decafluoro-3-methoxy-4-(trifluoromethyl)pentane (HFE-7300), ethyl nonafluoroisobutyl ether/ethyl nonafluorobutyl ether (HFE 8200), CHF₂OCHF₂, CHF₂—OCH₂F, CH₂F—OCH₂F, CH₂F—O—CH₃, cyclo-CF₂CH₂CF₂—O, cyclo-CF₂CF₂CH₂—O, CHF₂—CF₂CHF2, CF₃CF₂—OCH₂F, CHF₂—O—CHFCF₃, CHF₂—OCF₂CHF₂, CH₂F—O—CF₂CHF₂, CF₃—O—CF₂CH₃, CHF₂CHF—O—CHF₂, CF₃—O—CHFCH₂F, CF₃CHF—O—CH₂F, CF₃—O—CH₂CHF₂, CHF₂—O—CH₂CF₃, CH₂FCF₂—O—CH₂F, CHF2-O—CF₂CH₃, CHF₂CF₂—O—CH₃ (HFE254pc), CH₂F—O—CHFCH₂F, CHF₂—CHF—O—CH₂F, CF₃—O—CHFCH₃, CF₃CHF—O—CH₃, CHF₂—O—CH₂CHF₂, CF₃—O—CH₂CH₂F, CF₃CH₂—O—CH₂F, CF₂HCF₂CF₂—O—CH₃, CF₃CHFCF₂—O—CH₃, CHF₂CF₂CF₂—O—CH₃, CHF₂CF₂CH₂—OCHF₂, CF₃CF₂CH₂—O—CH₃, CHF₂CF₂—O—CH₂CH₃, (CF₃)₂CF—O—CH₃, (CF₃)₂CH—O—CHF₂, (CF₃)₂CH—O—CH₃, and mixture thereof; (V) C1 to C5 alcohols, C1 to C4 aldehydes, C1 to C4 ketones, C1 to C4 ethers and diethers and carbon dioxide; (VI) HCFOs such as, 1-chloro-3,3,3-trifluoropropenen (HCFO-1233zd), 2-chloro-3,3,3-trifluoropropene (HCFO-1233xf) and HCFO1223.

The foamable compositions of the present invention generally include one or more components capable of forming foam having a generally cellular structure and a blowing agent, typically in a combination, in accordance with the present invention. In certain embodiments, the one or more components comprise a polyurethane composition capable of forming foam and/or foamable compositions. In such polyurethane foam embodiments, one or more of the present compositions are included as or part of a blowing agent in a foamable composition, or as a part of a two or more part foamable composition, which preferably includes one or more of the components capable of reacting and/or foaming under the proper conditions to form a foam or cellular structure.

The invention also relates to foam, and preferably closed cell foam, prepared from a polymer foam formulation containing a blowing agent comprising the compositions of the present invention. In yet other embodiments, the invention provides foamable compositions comprising thermosetting foams, such as polyurethane and polyisocyanurate foams, preferably low-density foams, flexible or rigid, such as pour-in-place for insulation of refrigerated cavities, building and refrigerated panels, garage doors, entrance doors, insulated pipes, and water heaters; continuous lamination for metal and flexible faced panels; and spray for residential and commercial constructions and buildings.

It will be appreciated by those skilled in the art that the order and manner in which the blowing agent combination of the present invention is formed and/or added to the foamable composition does not generally affect the operability of the present invention. For example, in the case of polyurethane foams, it is possible that the various components of the blowing agent combination, and even the components of the present composition, not be mixed in advance of introduction to the foaming equipment, or even that the components are not added to the same location in the foaming equipment. Thus, in certain embodiments it may be desired to introduce one or more components of the blowing agent combination in a blender with the expectation that the components will come together in the foaming equipment and/or operate more effectively in this manner. Nevertheless, in certain embodiments, two or more components of the blowing agent combination are combined in advance and introduced together into the foamable composition, either directly or as part of premix that is then further added to other parts of the foamable composition.

In certain embodiments, b-side, polyol premixes may comprise polyols, silicon or non-silicon based surfactants, amine or non-amine based catalysts, flame retardants/suppressors, acid scavengers, radical scavengers, fillers, and other necessary stabilizers/inhibitors. Polyols may comprise Glycerin based polyether polyols such as Carpol GP-700, GP-725, GP-4000, GP-4520, and etc; Amine based polyether polyols such as Carpol TEAP-265 and EDAP-770, Jeffol AD-310, and etc; Sucrose based polyether polyol, such as Jeffol SD-360, SG-361, and SD-522, Voranol 490, Carpol SPA-357, and etc; Mannich base polyether polyol such as Jeffol R-425X and R-470X, and etc; Sorbitol based polyether polyol such as Jeffol S-490 and etc; Aromatic polyester polyols such as Terate 2541 and 3510, Stepanpol PS-2352, Terol TR-925, and etc.

Catalysts may comprise N,N-dimethylethanolamine (DMEA), N,N-dimethylcyclohexylamine (DMCHA), Bis(N,N-dimethylaminoethyl)ether (BDMAFE), N,N,N′,N′,N″-pentamethyldiethylenetriamine (PDMAFE), 1,4-diazadicyclo[2,2,2]octane (DABCO), 2-(2-dimethylaminoethoxy)-ethanol (DMAFE), 2-((2-dimethylaminoethoxy)-ethyl methyl-amino)ethanol, 1-(bis(3-dimethylamino)-propyl)amino-2-propanol, N,N′,N″-tris(3-dimethylamino-propyl)hexahydrotriazine, dimorpholinodiethylether (DMDEE), N,N-dimethylbenzylamine, N,N,N′,N″,N″-pentaamethyldipropylenetriamine, N,N′-diethylpiperazine, and etc. In particular, sterically hindered primary, secondary or tertiary amines are useful, for example, dicyclohexylmethylamine, ethyldiisopropylamine, dimethylcyclohexylamine, dimethylisopropylamine, methylisopropylbenzylamine, methylcyclopentylbenzylamine, isopropyl-sec-butyl-trifluoroethylamine, diethyl-(α-phenyethyl)amine, tri-n-propylamine, dicyclohexylamine, t-butylisopropylamine, butylamine, cyclohexyl-t-butylamine, de-sec-butylamine, dicyclopentylamine, di-(α-trifluoromethylethyl)amine, di-(α-phenylethyl)amine, triphenylmethylamine, and 1,1,-diethyl-n-propylamine. Other sterically hindered amines are morpholines, imidazoles, ether containing compounds such as dimorpholinodiethylether, N-ethylmolpholine, N-methylmorpholine, bis(dimethylaminoethyl)ether, imidizole, n0methylimidazole, 1,2-dimethylimidazole, dimorpholinodimethylether, N,N,N′,N′,N″,N″-pentamethyldiethylenetriamine, N,N,N′,N′,N″,N″-pentaethyldiethylenetriamine, N,N,N′,N′,N″,N″-pentamethyldipropylenetriamine, bis(diethylaminoethyl)ether, bis(dimethylaminopropyl)ether, or combination thereof.

Non-amine catalysts may comprise an organometallic compound containing bismuth, lead, tin, antimony, cadmium, cobalt, iron, thorium, aluminum, mercury, zinc, nickel, cerium, molybdenum, titanium, vanadium, copper, manganese, zirconium, magnesium, calcium, sodium, potassium, lithium, or combination thereof. Examples of such organometallic compound include stannous octoate, dibutyltin dilaurate (DGTDL), dibutyltin mercaptide, phenylmercuric propionate, lead octoate, potassium acetate/octoate, magnesium acetate, titanyl oxalate, potassium titanyl oxalate, quaternary ammonium formates, ferric acetylacetonate, and the like and combination thereof.

The use level of catalysts are typically in an amount of 0.1 ppm to 4.00 wt % of polyol premix, preferably from 0.5 ppm to 2 wt %, and more preferably from 1 ppm to 1 wt %.

The surfactants may comprise polysiloxane polyoxyalkylene block co-polymer such as B8404, B8407, B8409, B8462 and B8465 of Goldschmidt, DC-193, DC-197, DC-5582, and DC-5598 of Air Products, L-5130, L5180, L-5340, L-5440, L-6100, L-6900, L-6980, and L6988 of Momentive. Non-silicone surfactants may comprise salts of sulfonic acid, alkali metal salts of fatty acid, ammonium slats of fatty acid, oleic acid, stearic acid, dodecylbenzenedidulfonic acid, dinaphthylmetanedissulfonic acid, ricinoleic acid, an oxyethylated alkylphenol, an oxyethylated fatty alcohol, a paraffin oil, a caster oil ester, a ricinoleic acid ester, Turkey red oil, groundnut oil, a paraffin fatty alcohol, or combination thereof. The typically use levels are 0.4 to 6 wt % of polyol premix, preferably 0.8 to 4.5 wt %, and more preferably 1 to 3 wt %.

Flame retardants may comprise trichloropropyl phosphate (TCPP), triethyl phosphate (TEP), diethyl ethyl phosphate (DEEP), diethyl bis(2-hydroxyethyl)amino methyl phosphonate, brominated anhydride based ester, dibromoneopentyl glycol, brominated polyether polyol, melamine, ammonium polyphosphate, aluminium trihydrate (ATH), tris(1,3-diehloroisopropyl) phosphate, tri)-2-chlororthyl) phosphate, tri(2-chloroisopropyl) phosphate, chloroalkyl phosphate/oligomeric phosphonate, oligomeric chloroalkyl phosphate, brominated flame retardant based on pentabromo diphenyl ether, dimethyl methyl phosphonate, diethyl N,N bis(2-hydroxyethyl)amino methyl phosphonate, oligomeric phosphonate, and derivatives of above mentioned.

In certain embodiments, acid scavengers, radical scavengers, and other stabilizers/inhibitors are desired. Stabilizers/inhibitors may comprise 1,2-epoxy butane, glycidyl methyl ether, cyclic-terpenes such as di-limonene, 1-limonene, d-limonene, and etc, 1,2-epoxy-2,2-methylpropane, nitromethane, diethylhydroxyl amine, alpha methylstyrene, isoprene, p-methoxyphenol, m-methoxyphenol, dl-limonene oxide, hydrazines, 2,6-di-t-butyl phenol, hydroquinone, organic acids such as carboxylic acid, dicarboxylic acid, phosphonic acid, sulfonic acid, sulfamic acid, hydroxamic acid, formic acid, acetic acid, propionic acid, butyric acid, caproic acid, isocaprotic acid, 2-ethylhexanoic acid, caprylic acid, cyanoacetic acid, pyruvic acid, benzoic acid, oxalic acid, malonic acid, succinic acid, adipic acid, azelaic acid, trifluoroacetic acid, methanesulfonic acid, benzenesulfonic acid, and combination thereof. Other additives may comprise adhesion promoters, anti-static, antioxidant, filler, hydrolysis, lubricants, anti-microbial, pigments, viscosity modifiers, UV resistance additives, are also desired as needed. Examples of these additives include, but are not limited to, sterically hindered phenols, diphenylamines, benzofuranone derivatives, butylated hydroxytoluene (BHT), calcium carbonate, barium sulphate, glass fibers, carbon fibers, micro-spheres, silicas. Melamine, carbon black, form of waxes and soaps, organometallic derivatives of antimony, copper, and arsenic, titanium dioxide, chromium oxide, iron oxide, glycol ethers, dimethyl AGS esters, propylene carbonate, benzophenone and benzotriazole compounds derivatives.

### EXAMPLES

### Example 1

### Boiling Points and Solubility of Blowing Agents in Polyol Blends

TABLE 1
Boiling point and solubility in polyol blends

| Blowing agent | Boiling point (° C.) | Solubility |
|---|---|---|
| E-1233zd | 18 | ++ |
| HFC245fa | 15 | + |
| HCFC141b | 33 | +++ |
| Pentanes* | ** | − |

*Cyclo-pentane/iso-pentane = 80/20, cyclo-pentane boiling point: 49° C., and iso-pentane boiling point: 28° C.

Table 1 shows that E-1233zd has a boiling between HFC245fa and HCFC141b, and solubility also follows the same trend. The lower the boiling point, the higher vapor pressure, providing more expansion of foams and thus lower foam density. The solubility of blowing agent would affect the viscosity of polymer premix, the better the solubility, the lower the viscosity.

### Example 2

### Formulation and Reactivity

TABLE 2
Formulations

| | E-1233zd | HFC245fa | HCFC141b | Pentanes |
|---|---|---|---|---|
| B side | | | | |
| Voranol 490 | 17.39 | 17.35 | 17.60 | 18.25 |
| Jeffol R-425-X | 10.43 | 10.41 | 10.56 | 10.95 |
| Stepan 2352 | 6.96 | 6.94 | 7.04 | 7.30 |
| PMDETA | 0.16 | 0.16 | 0.16 | 0.16 |
| (PC5) | | | | |
| DMCHA (PC8) | 0.50 | 0.50 | 0.50 | 0.50 |
| Tegostab B8465 | 0.71 | 0.71 | 0.71 | 0.71 |
| TCPP | 2.36 | 2.36 | 2.36 | 2.36 |
| Blowing Agent | | | | |
| Water | 0.74 | 0.74 | 0.74 | 0.74 |
| E-1233zd | 8.04 | 0 | 0 | 0 |
| HFC245fa | 0 | 8.26 | 0 | 0 |
| HCFC141b | 0 | 0 | 7.20 | 0 |
| Pentanes | 0 | 0 | 0 | 4.34 |
| Total B Side | 47.29 | 47.42 | 46.86 | 45.32 |
| A Side | | | | |
| ROH Index | 115 | 115 | 115 | 115 |
| Isocyanate | 52.7 | 52.6 | 53.1 | 54.7 |
| A/B | 1.11 | 1.11 | 1.13 | 1.21 |
| Total Blowing | 23.0 | 23.0 | 23.0 | 23.0 |

TABLE 3
Reactivity and free rise density

| | E-1233zd | HFC245fa | HCFC141b | Pentanes |
|---|---|---|---|---|
| Chemical Temperature | 70/70 | 70/70 | 70/70 | 70/70 |
| (° F.) | | | | |
| Cream time (seconds) | 6 | froth | 5 | 5 |
| Gel time (seconds) | 28 | 29 | 29 | 27 |
| Tack free time | 43 | 46 | 45 | 44 |
| (seconds) | | | | |
| Core density in bucket | 1.69 | 1.65 | 1.65 | 1.68 |
| (pcf) | | | | |

Table 3 shows that the reactivities of four systems are quite similar to each other.

### Example 3

### Molding

The total B component and isocyanate were mixed using an Edge-Sweets 25 HP-BT high-pressure foam machine equipped with an L-head. Chemical temperatures were maintained at 70° F. prior to mixing at 1800 psi mix pressure and a total chemical throughput of 160 g/sec.

Molded foams were produced using a water jacketed aluminum mold commonly referred to as a Brett mold or Lanzen panel which measured 5 cm thick by 20 cm wide by 200 cm tall and were kept at a temperature around 115° F. A minimum fill density, i.e., just enough foam to fill the entire mold without any amount of packing, was first established using data (length the foam flowed and panel weight) from shots made at 2, 3, and 4 seconds. A panel was produced at 115% of the shot weight calculated for the minimum fill density.

In addition to determining the density of the panel, a comparison was made of the overall density versus the core density. This was done by taking every other 10 cm section along the length of the panel starting 60 cm from the bottom, measuring the density with the foam skin on, and then trimming off the skin to measure the core density. The data was compared to similar foams made with other blowing agents and found to give the least difference between overall and core densities.

TABLE 4
Minimum fill weight

| | Minimum fill weight | Minimum fill density |
|---|---|---|
| | (g) | (pcf*) |
| E-1233zd | 734 | 2.63 |
| HFC245fa | 727 | 2.60 |
| HCFC141b | 796 | 2.90 |
| Pentanes | 790 | 2.85 |

*pcf: lb/ft³

From Table 4, it can be seen that the minimum fill weight and density of E-1233zd foam fall between HFC245fa and HCFC141b. HCFC141b that has the best solubility in polyol blends does not have the lowest minimum fill weight. However, HFC245fa that has the lowest boiling point provides a foam with the lowest minimum fill density.

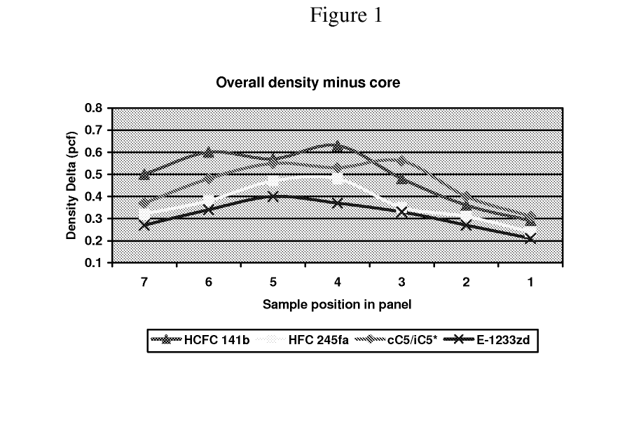

The results suggest that the boiling point has more influence on minimum fill weight than the solubility of a blowing agent. FIG. 1 showed how density varied in the flow path within the panel. It appeared that higher boiling point blowing agent such as HCFC141b and pentanes had more variation or less uniform distribution than lower boiling point blowing agent such as HFC245fa, and E-1233zd. Following the boiling point trend, one would predict that the density distribution of E-1233zd foam should fall between HFC245fa and HCFC141b. However, as shown in FIG. 1, E-1233zd foam showed the least variation of density or the most uniform form density, therefore, this result is not expected.

Although the invention is illustrated and described herein with reference to specific embodiments, it is not intended that the appended claims be limited to the details shown. Rather, it is expected that various modifications may be made in these details by those skilled in the art, which modifications may still be within the spirit and scope of the claimed subject matter and it is intended that these claims be construed accordingly.

1. Polyurethane foam with density variation, which is defined by overall density minus core, from 0.10 to 0.65 pcf, preferably from 0.15 to 0.50 pcf.

2. A method of producing polyurethane foam comprises (i) mixing blowing agent with other polyurethane premix components; (ii) high pressure mixing and dispensing equipment of reaction injection molding

3. A polymer blowing agent composition for polyurethane foams comprising about 70 wt % or more trans stereoisomer of hydrochlorofluoroolefin. 1233zd.

4. The polymer blowing agent composition of claim 3 wherein said hydrochlorofluoroolefin 1233zd comprises about 90 wt % or more trans stereoisomer.

5. The polymer blowing agent composition of claim 3 wherein said hydrochlorofluoroolefin 1233zd comprises about 97 wt % or more trans stereoisomer.

6. The polymer blowing agent composition of claim 3, further comprising a hydroflouorocarbon.

7. The polymer blowing agent composition of claim 6 wherein said hydrofluorocarbon is sleeted from the group consisting of difitioromethane (HFC32); 1,1,1,2,2-pentalluomethane (HFC125); 1,1,1-trifluoroethane (HFC143a); 1,1,2,2-tetrafluorothane (HFC 134); 1,1,1,2-tetrafluoroethane (HFC134a); 1,1-difluoroethane (HFC152a); 1,1,1,2,3,3,3-heptafluoropropane (HFC227ea); 1,1,1,3,3-pentafluopropane (HFC245fa.); 1,1,1,3,3-pentafluobutane (HFC365mfc) and 1,1,1,2,2,3,4,5,5,5-decafluoropentane (HFC4310mee).

8. The polymer blowing agent composition of claim 3, further comprising a hydrofluoroolefin.

9. The polymer blowing agent composition of claim 8 wherein said hydrofluoroolefin is selected from the group consisting of tetrafluoropropenes (HFO1234); trifluoropropenes (HFO1243); tetrafluorobutene isomers (HFO1354); pentafluorobutene isomers (HFO1345); hexafluorobutene isomers (HFO1336); heptafluorobutene isomers (HFO1327); heptafluoropentene isomers (HFO1447); octafluoropentene isomers (HFO1438); nonafluoropentene isomers (HFO1429), C4F9OCH3 (HFE-7100), C4F9OC2H5 (HFE-7200), CF3CF2OCH3 (HFE-245ch2), CF3CH2CHF2 (HFE-245fa), CF3CH2OCF3 (HFE-236fa), C3F7OCH3 (HFE-7000), 2-trifluoromethyl-3-ethoxydodecofluorobexane (HFE 7500), 1,1,1,2,3-hexafluoro-4-(1,1,2,3,3,3-hexafluoropropoxy)-pentane (HFE-7600), 1,1,1,2,2,3,5,5,5-decafluoro-3-methoxy-4-(trifluoromethyl)pentane (HFE-7300), ethyl nonafluoroisobutyl ether/ethyl nonafluorobutyl ether (HFE 8200), CHF2OCHF2, CHF2-OCH2F, CH2F—OCH2F, CH2F—O—CH3, cyclo-CF2CH2CF2-O, cyclo-CF2CF2CH2-O, CHF2-CF2CHF2, CF3CF2-OCH2F, CHF2-O—CHFCF3, CHF2-OCF2CHF2, CH2F—O—CF2CHF2, CF3-O—CF2CH3, CHF2CHF—O—CHF2, CF3-O—CHFCH2F, CF3CHF—O—CH2F, CF3-O—CH2CHF2, CHF2-O—CH2CF3, CH2FCF2-O—CH2F, CHF2-O—CF2CH3, CHF2CF2-O—CH3 (HFE254 pc), CH2F—O—CHFCH2F, CHF2-CHF—O—CH2F, CF3-O—CHFCH3, CF3CHF—O—CH3, CHF2-O—CH2CHF2, CH3-O—CH2CH2F, CF3CH2-O—CH2F, CF2HCF2CF2-O—CH3, CF3CHFCF2-O—CH3, CHF2CF2CF2-O—CH3, CHF2CF2CH2-OCHF2, CF3CF2CH2-O—CH3, CHF2CF2-O—CH2CH3, (CF3)2CF—O—CH3, (CF3)2CH—O—CHF2, (CF3)2CH—O—CH3, and mixture thereof.

10. The polymer blowing agent composition of claim 3, further comprising an additive selected from the group consisting of hydrocarbons, C1 to C5 alcohols, C1 to C4 aldehydes, C1 to C4 ketones, C1 to C4 ethers, carbon dioxide, C1 to C4 diethers, and mixture thereof.

11. The polymer blowing agent composition of claim 3, further comprising a hydrofluoroether.

12. The polyurethane foam of claim 1, with density variation, which is defined by overall density minus core, from 0.15 to 0.50 pcf.

13. The polyurethane foam of claim 1 with density variation, which is defined by overall density minus core, from 0.20 to 0.45 pcf.
